(19) United States
(12) Patent Application Publication
(10) Pub. No.: US 2025/0332316 A1
(43) Pub. Date: Oct. 30, 2025

(54) MULTI-LINEAR GRATING GUV LED FLOWING AIR AND FLUID STERILIZATION METHOD AND STERILIZATION SPACE DEVICE THEREOF

(71) Applicant: LightBeam One International co., Ltd., New Taipei City (TW)

(72) Inventor: Hsien-Sheng Lin, New Taipei City (TW)

(21) Appl. No.: 18/821,255

(22) Filed: Aug. 30, 2024

(30) Foreign Application Priority Data
Apr. 26, 2024 (TW) 113115753

(51) Int. Cl. A61L 9/20; A62B 18/00; A62B 23/02; A62B 23/06; C02F 1/32

(52) U.S. Cl. CPC A61L 9/20; A62B 18/006; C02F 1/325; A61L 2209/11; A61L 2209/12; A61L 2209/14; A62B 23/02; A62B 23/06; C02F 2201/3222; C02F 2201/3228; C02F 2201/326; C02F 2303/04

(57) ABSTRACT

The present invention relates to a multi-linear grating GUV LED flowing air and fluid sterilization method and a sterilization device thereof, multiple grating linear irradiation light of the GUV LED packages and the reflected light or retro-reflected light by the sterilization space chamber are linear and perpendicular to a cylindrical body in multiple times, and are not prone to overflow and pollute the environment in the same sterilization space quadrant. The retro-reflected light in the same quadrant forms reflected light in 360 degrees because of the repeated changes in direction, so that pathogens are exposed, increasing the chance of radiation overlapping. This forms the multi-linear grating GUV LED flowing air and fluid sterilization method and its sterilization space device of the present invention.

### BACKGROUND OF THE INVENTION

### Field of the Invention

The present invention relates to a multi-linear grating GUV (germicidal ultraviolet light) LED flowing air and fluid sterilization technology, and specifically, to a multi-linear grating GUV LED flowing air and fluid sterilization method and its sterilization space device for rapid disinfection, sterilization, and high-flow decontamination of air and fluid.

### Description of the Prior Art

Ultraviolet light-emitting diodes (UV LEDs), which can emit ultraviolet rays invisible to naked eyes, have been used as a powerful disinfection and sterilization instrument, for example, to disinfect drinking water or tableware. The use of ultraviolet radiations can achieve efficient disinfection without using drugs or chemicals. This method is safe and is free of secondary pollution such as assimilable organic carbon (AOC) and dissolved organic carbon (DOC), making it an ideal disinfection method.

However, the currently known UVC LED devices for disinfection and sterilization still have several drawbacks: 1. firstly, not all ultraviolet rays are effective in disinfection and sterilization; the ultraviolet light has to be in the absorption wavelength range of pathogen's DNA or RNA so that this ultraviolet light can trigger chemical reaction with the absorption of light quanta 2. the absorbed radiation dose must accumulate to a level sufficient to cause DNA or RNA cleavage or strand breakage for the pathogens to be inactivated, lose infectivity, or be decomposed and eliminated. Therefore, the DNA or RNA absorption wavelength range of 234˜313 nm, which includes UVB and a small portion of UVC, is the most effective sterilization range, called UGV (Germicidal ultraviolet) radiation sterilization light.

UV LEDs also have the following drawbacks:
a. a light-emitting angle θ of UV LED: the LED light intensity angle from the peak to where it attenuates to 50%, that is, the uniformity tolerance is above 50%, results in large differences in radiation dose depending on the environment. Therefore, it is difficult to increase the sterilization rate as some pathogens may escape radiation.;
b. The general LED projected light is a 3D divergent cone with a large radiation projection area, which leads to scattering of radiation and makes the radiation dose intensity per unit area weaker. As a result, this radiation cannot effectively purify fast flowing air or fluid. Since K=I×t, when the radiation dose (I) is lower, the time (t) needs to be increased to accumulate the pathogen inactivation dose level (K);
c, an uncontrolled radiation exposure angle may cause ultraviolet rays to leak outside the disinfection area, resulting in environmental pollution by ultraviolet light, which will cause harm to human body upon contact;
d. Currently, most UV LED disinfection and sterilization devices are designed as parallel to the flow direction of flowing air and fluid, and the radiation intensity is inversely proportional to the square of distance. This results in poor efficiency in radiation energy accumulation, making it difficult to quickly accumulate sufficient lethal dose of radiation intensity, thus causing a lower flowing air and fluid processing efficiency; and
e. Lack of effective use of reflected light: the current UV LED design is mainly based on a matrix arrangement with radiations parallel to the fluid, and there is no reflection design. Therefore, the radiation is used once only, before dispersing, resulting in waste of radiation energy.

Therefore, for non-flowing air and fluid sterilization, such as surface sterilization of static objects. The radiation intensity can be accumulated over time to solve the batch processing issue. Thus, there is not a huge issue with long-term irradiation sterilization on static objects. However, for flowing fluid like air or water that need continuous flow processing, a large volume needs to be treated in a short period of time. Since the flowing fluid could not stay static for accumulating sufficient radiation energy, the sterilization rate becomes problematic. Moreover, flowing air and fluid disinfection is always a continuous process that needs to be done in an open space. If the light outflow cannot be controlled, the radiation can easily harm human skin and eyes.

The ultraviolet light from existing UV LED disinfection and sterilization devices has uneven radiation intensity and insufficient strength, unable to meet the requirements for rapid and high-volume sterilization of flowing air and fluid. Therefore, there is still an urgent need of highly efficient and safe technology for this issue.

The present invention of multi-linear grating GUV LED method for flowing air and fluid sterilization and the sterilization space device controls the flowing fluid sterilization operation within a sterilization space chamber. Inside, there is an optical package design with intensified linear grating radiation. It uses 2D linear grating sterilization light, irradiating from the outside inward onto materials with high UV reflectivity. By utilizing cylindrical linear grating reflection or anti-reflection radiation coexisting in the same quadrant plane, it increases the chances of radiation overlap. This enhances the radiation dose within the sterilization space and shortens sterilization time. Due to the controlled radiation direction of the linear reflective mirror device, it is not prone to leaking out of the sterilization space, making it environmentally friendly and preventing secondary environmental pollution issues.

### SUMMARY OF THE INVENTION

To overcome the aforementioned shortcomings, the main objective of the present invention is to utilize a radiation light path design involving two Maddox Rods. The first round transforms the GUV LED radiation through a cylindrical lens, forming a linear 2D sterilization grating projection light perpendicular to the direction of the cylindrical lens. This light is then introduced into the sterilization space through a window. The second Maddox Rod involves reflection and anti-reflection from the cylindrical mirror on the inner surface of the sterilization space, resulting in multi-linear grating GUV LED radiation light.

The present invention's next objective is the flowing air and fluid sterilization method, which utilizes a hollow cylindrical tube to form an effective sterilization space chamber. This allows water to flow through the sterilization space for water sterilization, serving as a water sterilization space chamber.

Another objective of this invention is the flowing air sterilization method, in which the air flows through the sterilization space device for air sterilization, serving as an air sterilization device.

Yet another objective of this invention is to deliver the sterilized clean air into the space between masks, oral-nasal mask, face shields and the nose, forming an application as a positive pressure mask.

To achieve the aforementioned objectives, the present invention provides a linear grating GUV LED flowing air and fluid sterilization method and its sterilization space device. The linear grating GUV LED packages possess an arc-shaped cylindrical lens encapsulation. This arc-shaped cylindrical lens is a cylindrical body lens with a concave arc, convex arc, continuous concave cylindrical, or continuous convex cylindrical shape. The UV light emitted by the linear grating GUV LED package passes through the arc-shaped cylindrical lens encapsulated at the end face.

The radiation passes through the plane perpendicular to the cylinder axis, with the divergence rate maintained unchanged and the unequal refraction undergoes in other planes. This ultimately forms light rays perpendicular to the cylinder axis direction. These parallel light rays extend outward to form light planes, and these numerous parallel light planes are called linear grating planes. While traditional emission light disperse in a 3D spherical manner, the arc-shaped cylindrical lens enable the formation of 2D radiation light rays in the vertical plane. These light rays form numerous parallel light planes in the cylindrical vertical plane, similar to linear grating cross-sections. The radiation intensity is inversely proportional to the square of the distance, and there is a significant difference in the distance-area ratio between 2D and 3D manners.
The grating GUV LED radiation intensity in the present invention is much higher than general UVC packages. The radiation light is emitted linearly through a pre-reserved window on the side of the sterilization space device, radiating from the outside inward into the interior of the sterilization space chamber. The light incident from the lateral vertical cross-section is designed in its second light path to irradiate onto a highly reflective cylindrical body. When the reflected light is reflected again by the continuous cylindrical mirror on the inner surface of the sterilization space chamber, its path will be perpendicular to the continuous cylindrical mirror chamber, just like a linear grating surface.
The projected light, reflected light, or anti-reflected light coexist in the same quadrant as multiple parallel linear grating surface lights, increasing the probability of radiation overlap. The radiation is intensified due to the superposition. The radiation reflects as if in a closed environment. Based on the Sumpner radiation principle for closed systems, calculating with aluminum's reflectivity R=90% for the UVC band: E=ED+ER, where ED is the original irradiance, and ER is the reflected irradiance. ER=ED×(R/(1−R)), calculating E total radiation=1 (original light source)+9 (superposition of reflections)=10. This configuration is equivalent to the intensification to 10 times of GUV radiation. The reuse of reflected radiation light is beneficial for fast sterilization. The radiation uniformity becomes more uniform due to superposition. The repeated use of highly reflective light results in high radiation intensity and high sterilization efficiency, which is the first function of this invention's device. The 2D linear grating light surface makes the radiation direction controllable. The radiation only reflects in the vertical direction of the cylindrical mirror, towards the direction of the cylindrical cross-section. This controlled radiation direction is not prone to outflow and polluting the environment, which is the second function of this invention's device.
Another advantage of UV sterilization is that no UV-resistant viruses or bacteria have been discovered on Earth yet. Radiation traveling in straight lines, while bacteria may easily hide underneath. With the current invention, the projected light, reflected light, and anti-reflected light are in the same quadrant, while the incident angle of the reflected radiation keeps changing direction, as well as the anti-reflected light. This solves the shortcoming of light only traveling in straight direction at fixed angles. This invention's method provides radiation at omnidirectional angles, leaving no place for bacteria to hide and thus being eliminated. Therefore, it increases the sterilization rate. This is the third function of this invention's device.
Not all UVC are able to eliminate pathogens. Microorganisms are classified into those with and without envelopes. Because the enveloped material is protein, its absorption peak at 220 nm allows the sterilization by ultraviolet light. Such UVC is ineffective for non-enveloped bacteria. The current invention's GUV utilizes the DNA absorption wavelength range (234˜313 nm) for DNA, which is the best sterilizing ultraviolet light GUV for all bacteria with. This is the fourth function of the current invention. These are all the functional features of this invention's method.

The linear grating GUV LED packages of this invention have their end face encapsulated by an arc-shaped cylindrical lens. This arc-shaped cylindrical body can be one of the following encapsulated lens: a convex arc-shaped cylindrical body, a concave arc-shaped cylindrical body, a continuous convex arc-shaped cylindrical body, or a continuous concave arc-shaped cylindrical body.

The cylindrical lens material of the linear grating GUV LED package in the present invention can be made of one of the following UV-transmitting materials: quartz, fluoropolymer, polydimethylsiloxane (PDMS), polyimide (PI), etc., forming the arc-cylindrical lens-encapsulated GUV LED linear grating package.

In another embodiment of the present invention, it includes any window that allows ultraviolet light to irradiate from the outside into the high sterilization space chamber. This window can be made of quartz that is permeable to ultraviolet light, or it can be a hole, which can be connected to the linear grating GUV LED package. The linear grating radiation enters the sterilization space chamber from the side. The material of this chamber is a hollow quartz tube, with its outer wall wrapped with highly reflective aluminum, or a vacuum-deposited aluminum layer on the outside of the quartz tube with only the window left to allow the GUV LED to pass through, or it can be made of highly reflective aluminum metal.

The radiation entering from the side window hits the highly reflective cylindrical body, forming multiple linear grating reflection layers once again. Both these reflection layer and anti-reflection layer are linear grating irradiation, with the radiation existing in the same quadrant, increasing the chance of radiation overlap, intensified radiation light and providing good light uniformity. This forms the sterilization space device.

Regarding the multi-linear grating GUV LED flowing air and fluid sterilization device in the present invention, it includes one or more linear grating GUV LED packages, which modify the GUV LED sterilization light into 2D linear transmission to enhance radiation intensity. In at least one embodiment, the linear grating GUV LED package of the present invention can be connected in series, in parallel, or configured into an array. Their DC power supply device can be derived from a general power source or a battery power source under 24 V, introduced into a control PCB board for power supply, used to light up the linear grating GUV LED packages or power the exhaust fan.

Regarding the concave arc-shaped linear grating GUV LED packages in the current invention, a window is reserved on the outside of the quartz tube. The hollow quartz tube is joined with the concave arc-shaped cylindrical end of the arc-shaped cylindrical lens, making the horizontal axis of the sterilization space chamber parallel to the horizontal axis of the arc-shaped cylindrical lens. Except for the curved end of the arc-shaped cylindrical lens which is joined as a transparent window on the outer wall, the rest of the outer wall of the quartz tube has an aluminum metal ultraviolet reflective surface coating or a vacuum-deposited aluminum reflection layer. The sterilization space chamber has openings at both ends to allow air or fluid to flow through the sterilization space chamber, serving as a multi-linear grating GUV LED flowing air and fluid disinfection and sterilization space device.

The linear grating GUV LED package of the present invention is connected to the sterilization space chamber, which is made of aluminum metal material. The inner surface of the hollow aluminum tube wall is either a continuous convex or continuous concave arc-shaped cylindrical reflective mirror, with openings at both ends. The inner hollow tube wall can be circular, rectangular, or square, with an inner diameter or hole diameter within 30 mm. There is a hole reserved on one side to serve as the window for installing the linear grating GUV LED package. There can be one or more holes of window. The linear grating GUV LED packages radiate through this hole into the sterilization space chamber, forming a grating radiation surface perpendicular to the cross-section of the cylindrical reflective mirror from the outside inwards. The phases of the reflected or anti-reflected radiation are different, but all are perpendicular to the cylindrical cross-section, located in the same cross-sectional quadrant. This increases the probability of radiation superposition, making the beam intensity stronger and more uniform, serving as a multi-linear grating GUV LED flowing air and fluid sterilization space chamber.

As in one of the embodiments mentioned above, a transparent quartz tube is fitted inside the hollow aluminum tube, serving as a window for installing the linear grating GUV LED package. The linear grating GUV LED package radiate through this hole into the quartz tube, then enter the sterilization quartz tube space chamber, irradiating from the outside inwards, forming a grating radiation surface perpendicular to the cross-section of the cylindrical reflective mirror. The reflected or anti-reflected radiation is reflected by the outer aluminum continuous cylindrical reflective mirror, entering the sterilization space again. The phases of the reflected light and anti-reflected light are different, but all are perpendicular to the cylindrical cross-section, located in the same cross-sectional quadrant. This increases the probability of radiation overlap, making the beam intensity stronger and more uniform. This serves as a flowing water and fluid sterilization space chamber, with the inner diameter of the chamber limited to within 30 mm. GUV is a high energy but with a low penetration. Water contains ions such as calcium, magnesium, silicon, oxygen, nitrogen, etc., which greatly affect GUV penetration. To ensure the sterilization efficiency, this limitation is added.

The present invention's linear grating GUV LED package device includes multiple linear grating GUV LED packages. There can be one or more packages, and these multiple GUV LED packages can be placed on the same side or different side of the sterilization space to increase the radiation intensity.

The radiation light wavelength from this invention's linear grating GUV LED package ranges between 234 nm and 313 nm. According to Einstein's light quantum theory, the cleavage of bacteria's DNA or RNA is related to their light absorption band. Only light quanta that can be absorbed can undergo energy conversion for chemical action, which destroys and cleaves the chemical bonds of DNA or RNA. Bacteria with broken DNA or RNA bonds will lose activity, which is referred to as disinfection and sterilization. According to the International Commission on Illumination (CIE), the ultraviolet light absorption peak is at 265 nm, with troughs at 234-313 nm. The present invention's linear grating ultraviolet sterilization band is 234-313 nm, termed as GUV LED. It includes portion of the UVC and part of the UVB wavelengths. With the modification by a cylindrical lens, the radiation light is corrected into a linear form based on the Maddox Rod transmission principle. The light source is not a single point. When multiple point light sources form multiple parallel linear projections, it is called linear grating projection, leading to this invention of linear grating GUV LED package.

The present invention also includes a device that utilizes the aforementioned linear grating GUV LED flowing air and fluid sterilization method. In at least one embodiment, it also includes a fan system and a front filter set up in the system, which actively sucks in air and passes it through the sterilization space chamber with multiple linear grating GUV LED packages. The air first passes through the filter to remove particulates, then enters the hollow chamber of the sterilization space, where it is sterilized by ultraviolet exposure. The clean air then enters masks, respirators, nasal masks, face shields, head covers, or bodysuits through nasal tubes, forming a positive pressure mask for people to use.

In other embodiments, the present invention's sterilization device is set up at the exhaust port of the protective cover. The protective cover is made of non-breathable polymer, with an air or oxygen inlet, creating positive pressure inside the protective cover. The used air is discharged from this exhaust port. Before the waste gas leaves the protective cover, it must first pass through the multi-linear grating GUV LED flowing fluid sterilization device. The sterilized air is then returned to the environmental space, creating an environment free of polluting pathogens.

The present invention also provides a linear grating GUV LED flowing air and fluid sterilization method and its sterilization space device. In at least one embodiment, this flowing fluid sterilization space device is used for water fluid sterilization. For example, it can be configured or combined with faucets, water dispensers, water outlets, drains, sewage treatment units, waste liquid treatment units, and liquid processors for medical waste.

In at least one embodiment, the fluid processor also includes a filter set up at the upstream end of the disinfection and sterilization space. Therefore, the GUV LED device can be used as an air processor for disinfectors, sterilizers, purifiers, or sterilizers.

This invention is a multi-linear grating GUV LED flowing fluid sterilization method. The steps include: 1) Activate the DC power supply device: This power unit can be a general power supply first rectified to below DC24V or a battery, with power sent to the control power PCB board via power supply contacts. 2) Linear grating GUV LED packages: Light up these linear grating GUV LED packages, emitting ultraviolet radiation of 234˜313 nm, which are made to emit in parallel through a cylindrical lens, forming a linear grating surface. 3) Radiation window device: This window device is on the side of the sterilization space chamber; it can be a quartz window permeable to GUV or a hole reserved for GUV LED radiation to enter, with radiation irradiating from outside to inside, entering the sterilization space chamber perpendicularly. 4) Sterilization space chamber: This device has openings at both ends, with a cylindrical reflective mirror on the inner surface, and a radiation entry window reserved on the side. GUV radiation light undergoes multiple linear grating reflections and anti-reflections on the inner surface of the sterilization space chamber. The fluid to be processed enters the sterilization space chamber from one end following the flow direction for sterilization exposure, and the sterilized fluid is discharged from the exit end of the sterilization space chamber.

The linear grating GUV LED flowing air and fluid sterilization radiation enters the sterilization space chamber from the side, with its reflection path perpendicular to the cylindrical structure of the sterilization space chamber. Therefore, the direction of ultraviolet ray inside the sterilization space form parallel linear grating surface perpendicular to the cylinder axis. Through repeated reflections within the cylindrical chamber, all reflections are in the same quadrant, increasing the probability of radiation superposition, thus increasing radiation intensity. Due to multiple superpositions, radiation uniformity is also improved accordingly. Moreover, as the ray paths are all perpendicular to the chamber cylinder, ultraviolet rays are confined within the sterilization space chamber, preventing ultraviolet ray outflow and secondary radiation contamination of the environment.

The current invention's technology is applied in sterilization treatment devices for flowing fluid such as fluid from faucets, water dispensers, water outlets, drains, sewage treatment units, waste liquid treatment units, and medical waste liquid, or for breathing air or oxygen, or medical waste gase.

The function of the current invention's sterilization space device is to allow air or fluid to pass through and have the GUV LED sterilization light operating within the sterilization space without leaking ultraviolet radiation to pollute the environment. GUV LED sterilization light can sterilize without causing drug resistance. However, it may harm human skin and eyes. Therefore, the best approach when applying GUV LED sterilization light is to have sterilization operating inside the sterilization space device, without exposing the GUV LED sterilization light to the air, causing harm to human body.

For air or water that needs continuous processing, the short processing time and high flow rate, as well as the ability to confine the GUV LED sterilization light outflow in a defined open space, are features of the current invention. In this invention's sterilization space device, the GUV LED sterilization light source first undergoes a primary correction through a cylindrical lens, making the light perpendicular to the arc-shaped cylindrical lens, forming a linear grating emission. It then reaches the window on the side of the fluid sterilization space chamber, radiating from the outside inward into the chamber. The GUV LED sterilization light then undergoes a correction along the direction of secondary reflective cylindrical linear grating, forming multiple parallel linear grating surfaces within the sterilization space chamber. Due to the transverse cylindrical surface reflection of the GUV LED sterilization light in the sterilization space chamber, each grating surface changes direction due to different incident angles of reflection, and the direction of the anti-reflected grating surface also changes. However, the reflection direction of the light always intersects the cylindrical direction and remains parallel of each other, which is called GUV LED multiple linear grating surface.
The GUV LED sterilization light, after multiple anti-reflections within the same quadrant, leads to the increase in probability of radiation superposition, making the radiation more uniform and increasing the radiation intensity. When the GUV LED sterilization light is reflected inside the chamber, the reflected light angle changes constantly with different incident angles, forming 360-degree GUV light coverage. This leaves no hiding place for viruses and bacteria under dusts, avoiding any missed pathogens. This is the function of the sterilization space device in the current invention's methodology.

Regarding the present invention's multi-linear grating GUV LED flowing and air fluid sterilization method and its sterilization space device, the property of air disinfection is utilized as the following:

The processed air is transported by a fan mechanism through nasal tubing into face shields, masks, or nasal masks. The clean air enters and creates positive pressure around the mouth and nose area, which can prevent viruses from entering the mouth and nose due to negative pressure. The positive pressure oxygen helps oxygen enter the lungs, while the high concentration of carbon dioxide and waste heat exhaled from the lungs can be quickly expelled out of the mask due to the positive pressure. This creates a protective function that keeps the wearer safe and comfortable.

Furthermore, for an non-breathable airtight face mask, oral mask, and nasal mask collectively known as a protective cover, a sterilization space device with the multi-linear grating-type GUV LED sterilization method of the present invention is mounted at its exhaust air outlet, so that the exhaust air containing viruses and bacteria of the wearer can be treated and sterilized before being discharged, thereby preventing it from infecting other people, for example, patients with a cold, tuberculosis, avian influenza, and COVID, and so forth.

In the multi-linear grating GUV LED sterilization method of the present invention and the sterilization space device thereof, due to its disinfection and sterilization properties for water fluid treatment, the sterilization space device is applied to the drinking water outlet, serving as a flowing water treatment device. Furthermore, the sterilization space devices are connected in parallel to increase the water treatment capacity and are used for cleaning food ingredients and bathing in household. Furthermore, it can be mounted in a drainage outlet where contamination is a concern, such as in medical wastewater for disinfection and inactivation, e.g., dental mouthwash to reduce viral and bacterial contamination.

### BRIEF DESCRIPTION OF THE DRAWINGS

This invention can be implemented in various ways. The following are specific explanatory but non-limiting examples of the implementation, which are also illustrated by the accompanying figures:

FIG. 1A is a schematic diagram of a linear grating GUV LED package comprised of a concave arc-shaped cylindrical lens;

FIG. 1B is a schematic diagram of a linear grating GUV LED package comprised of continuous concave arc-shaped cylindrical lenses;

FIG. 1C is a schematic diagram of a linear grating GUV LED package comprised of a convex arc-shaped cylindrical lens;

FIG. 1D is a schematic diagram of a linear grating GUV LED package comprised of continuous convex arc-shaped cylindrical lenses;

FIG. 2 is a schematic diagram of a flowing fluid sterilization space device of the present invention;

FIG. 3A is a cross-sectional schematic diagram of the present invention with an internal cylindrical flowing fluid sterilization space device;

FIG. 3B is a cross-sectional schematic diagram of the invention with an internal circular continuous arc convex columnar flowing fluid sterilization space device;

FIG. 3C is a cross-sectional schematic diagram of the present invention with an internal circular continuous arc concave columnar flowing fluid sterilization space device;

FIG. 3D is a cross-sectional schematic diagram of the present invention with an internal square continuous arc convex columnar flowing fluid sterilization space device;

FIG. 3E is a cross-sectional schematic diagram of the present invention with an internal square continuous arc concave columnar flowing fluid sterilization space device;

FIG. 3F is a cross-sectional schematic diagram of the flowing fluid sterilization space device combining a transparent internal cylinder with an internal highly reflective circular continuous arc concave column;

FIG. 4 is a schematic diagram of the application of present invention's flowing air sterilization space device in car air conditioning sterilization.;

FIG. 5 is a schematic diagram of the application of present invention's flowing air sterilization space device in an open oral mask for sterilization purpose.;

FIG. 6 is a schematic diagram of the present invention used for active flowing air sterilization application;

FIG. 7 is a schematic diagram of the present invention in the use as a positive-pressure face mask with flowing air sterilization

FIG. 8 is a schematic diagram of the present invention in the use as a positive-pressure oral mask with flowing air sterilization;

FIG. 9 is a schematic diagram of the present invention in the use as a positive-pressure nasal mask with flowing air sterilization;

FIG. 10 is a schematic diagram of the present invention's parallel multi-tubular air and fluid sterilization space device; and

FIG. 11 is a flowchart of a linear grating GUV LED air and fluid sterilization method of the present invention.

REFERENCE SIGNS

| | | | |
|---|---|---|---|
| 10 | Linear grating GUV | 20 | Multi-linear grating |
| | LED package | | GUV LED flowing air |
| | | | and fluid sterilization |
| | | | space device |
| 41 | Air outlet window | 42 | Air inlet |
| 100 | Lead frame substrate | 101 | GUV LED die |
| 102, 202, | Arc-shaped cylindrical | 103 | Sterilization space |
| 302, 402 | lens | | chamber |
| 106 | GUV LED fixed circuit | 107 | Control power PCB |
| | board | | board |
| 108 | Quartz tube | 109 | Flow direction |
| 110 | Aluminum-coated | 111 | Outer frame |
| | reflective layer | | |
| 112 | Power supply contact | 113 | Battery |
| 114 | Outlet end | 115 | Quartz window |
| 116 | Hole | 200 | Passive multi-linear |
| | | | grating GUV LED |
| | | | sterilization space |
| | | | device |
| 800 | Active multi-linear | 801 | Nasal cannula |
| | grating GUV LED | | |
| | sterilization space | | |
| | device | | |
| 802 | Fan | 803 | Air filter |
| 804 | Face mask | 805 | Face mask |
| 806 | Nasal mask | 808 | Air vent |
| 1000 | Protective cover | 1001 | Opening end |
| 1002 | Exhaust port | 1003 | Crumpled air |
| | | | expansion tank |
| 1004 | Air inlet port | 1081 | hollow tube permeable |
| | | | to GUV |
| 10-1, 10-2 | Multi-way tube joint | 1082-1085 | Continuous arc-shaped |
| | module | | cylindrical reflector |
| | | R | UVC reflectance |
| | | E | Total radiation |
| | | ED | Original radiation |
| | | | intensity |
| | | ER | Reflected radiation |
| | | | intensity |

### DETAILED DESCRIPTION OF THE PREFERRED EMBODIMENTS

With respect to the multi-linear grating GUV LED flowing air and fluid sterilization method and its sterilization space device claimed in the present invention, to enable a person having ordinary knowledge in the art to fully understand the objectives, features, and functions of the present invention, appropriate embodiments are set forth below and technical contents of the present invention are described in detail in conjunction with the attached drawings.

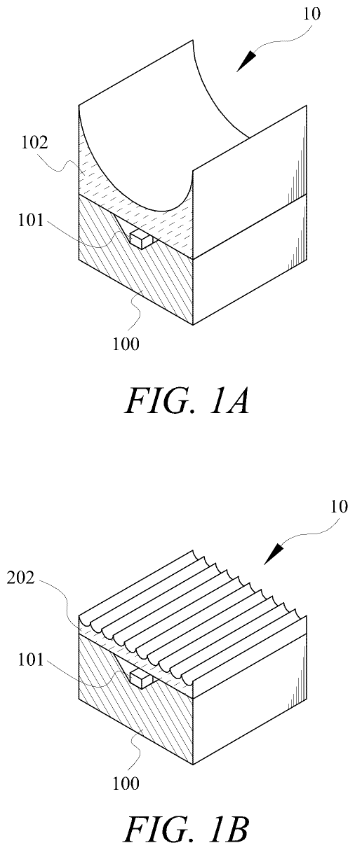

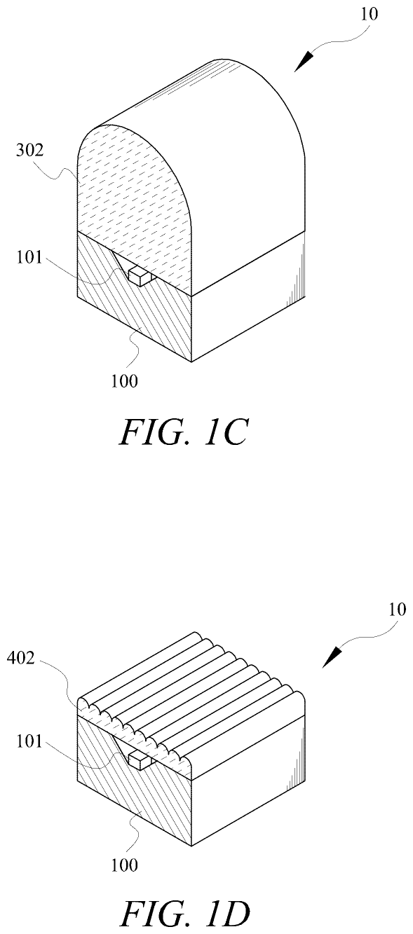

Referring to FIGS. 1A, 1B, 1C, and 1D for the cross-sectional schematic diagrams of the linear grating GUV LED package 10 set up in this embodiment. As shown in FIG. 1A, it includes a GUV LED die 101 encapsulated on a lead frame substrate 100. The sterilization light wavelength emitted by this die 101 is in the 234-313 nm ultraviolet radiation band. A concave arc-shaped cylindrical lens 102 made of ultraviolet-transmitting material is placed on top of the encapsulated GUV LED die 101. Alternatively, as shown in FIG. 1B, in another embodiment, the GUV LED package 101 is similarly encapsulated on the lead frame substrate 100, but a continuous concave arc-shaped cylindrical lens 202 is encapsulated on the die 101. Furthermore, as shown in FIG. 1C, a convex arc-shaped cylindrical lens 302 made of ultraviolet-transmitting material is encapsulated on GUV LED die 101. And, as shown in FIG ID, a continuous convex arc-shaped cylindrical lens 402 is encapsulated on GUV LED die 101.

Each of aforementioned arc-shaped cylindrical lenses 102, 202, 202, 402 is made of one of arc-shaped cylindrical ultraviolet-transmitting materials, such as quartz, sapphire, fluorinated polymer, polydimethylsiloxane (PDMS), and polyimide (PI). According to the Maddox Rod light transmission principle, the arc-shaped cylindrical lenses 102, 202, 302, 402 used for encapsulation will form linear light that spreads out on both sides perpendicular to the cylindrical axis of these lenses. This confines the GUV LED emission angle into parallel linear 2D sterilization light, extending outward to form a grating surface. This allows multiple parallel illumination surfaces to radiate as a linear grid, like a grating. The length of the radiation distance is determined by the curvature of the arc-shaped cylindrical lenses 102, 202, 302, 402. In combination with the lead frame substrate 100, the GUV LED die 101 is encapsulated to form what this invention calls a linear grating GUV LED package 10.

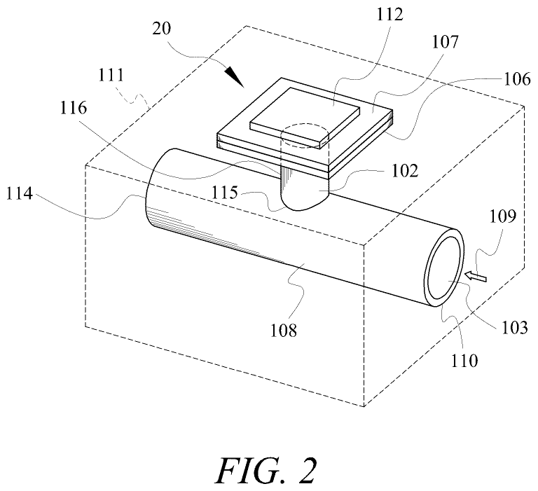

Referring to FIG. 2, it shows a schematic diagram of an embodiment of the linear grating GUV LED air and fluid sterilization space device 20 of the present invention. The linear grating GUV LED flowing air and fluid sterilization space device includes an outer frame 111 that can be made from a metal aluminum or a polymer material with aluminum powder added; a window hole 116 is placed in one side of the outer frame 111, which accommodates a linear grating GUV LED package 10 having a concave arc-shaped cylindrical lens 102 (as shown in FIG. 1A). The linear grating GUV LED flowing air and fluid sterilization space device 20 includes a GUV LED circuit board 106 for fixing, which is fixed on the circuit board 106 and fixed with a control power PCB 107 for managing the power supply; the power supply contact 112, which is connected to a DC power supply or a battery 113 as described later as a power supply, is located on the control power PCB 107. The concave arc-shaped cylindrical lens 102 of the linear grating GUV LED package 10 is provided on one side of the outer tube wall of the quartz tube 108. Except for a quartz window 115 as the joint portion between the concave arc-shaped cylindrical lens 102 and the quartz tube 108, an entire outer wall surface of the quartz tube 108 is coated with an aluminum powder reflective layer 110, or is vacuum-plated with aluminum metal, serving as a concave cylindrical reflector for radiation. The radiations will form a linear grating surface parallel to the cylindrical cross-section due to the reflection of the cylindrical quartz tube 108, and multiple back-and-forth retro-reflections increase the radiation intensity. The hollow cavity inside the quartz tube 108 is used as a sterilization space chamber 103 with a diameter of less than 30 mm to allow air and fluid to pass, therethrough as a sterilization space. The quartz tube has openings at both ends to allow the air and fluid to flow into the sterilization space chamber 103 of the quartz tube 108 in the direction 109, and the sterilized air and fluid flow out of an outlet end 114, so that the fluid sterilization space device serves as a disinfection and sterilization device for flowing water, drinking water, sewage, medical wastewater, and so on, and also is used for air sterilization.

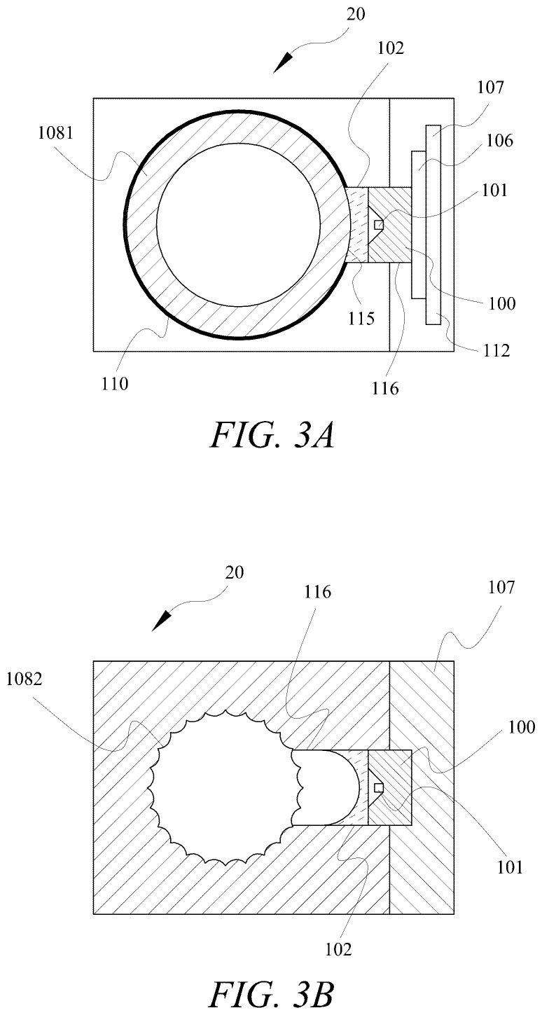

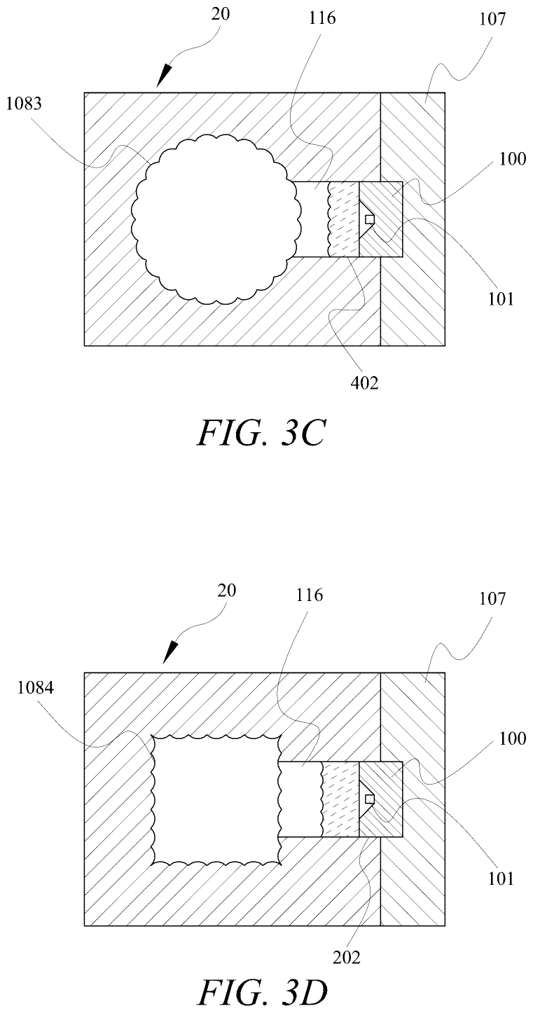

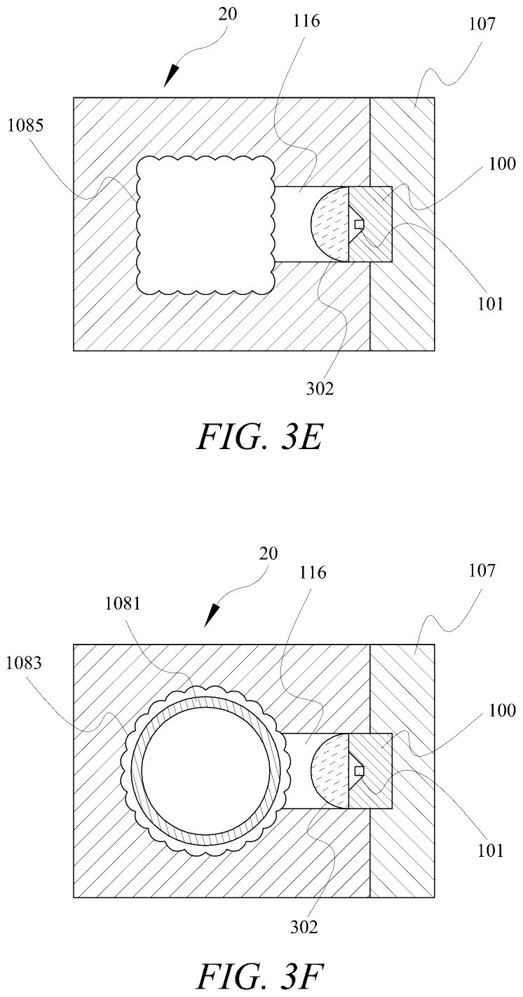

Referring to FIGS. 3A, 3B, 3C, 3D, 3E, 3F, sectional views of the air and fluid sterilization space device according to the present invention are shown. As shown in FIG. 3A, the linear grating GUV LED flowing air and fluid sterilization space device 20 has a structure in which the inner cylindrical arc-shaped cylindrical reflector 1081 made of a quartz material is plated with highly reflective aluminum to form a sterilization space chamber; or as shown in FIG. 3B, it uses metal aluminum or polytetrafluoroethylene (PTFE) processed into an internal circular shape, with a continuous convex columnar arc-shaped cylindrical reflector 1082 to form a sterilization space chamber; or as shown in FIG. 3C, it uses metal aluminum processed into an internal circular shape, with a continuous concave columnar arc-shaped cylindrical reflector 1083, to form the sterilization space chamber; or as shown in FIG. 3D, it uses metal aluminum or polytetrafluoroethylene processed into an internal square shape, with a continuous convex columnar arc-shaped cylindrical reflector 1084 as the sterilization space chamber; or as shown in FIG. 3E, it uses metal aluminum or polytetrafluoroethylene processed into an internal shape, with a continuous concave columnar arc-shaped cylindrical reflector 1085 as the sterilization space chamber; and as shown in FIG. 3F, it combines the inner cylindrical arc-shaped columnar reflector as shown in FIG. 3C with a hollow quartz tube or hollow fluoropolymer tube 1081 that is permeable to GUV. This component 1081 can be moved or replaced. It is combined with the continuous concave columnar arc-shaped columnar reflector 1083 as shown in FIG. 3C to form a sterilization space chamber, and so on; one of these sterilization space cavities is implemented, and the component structure of the linear grating GUV LED package 10 is installed through the GUV LED hole 116, or a pre-reserved quartz window 115. The depth or width of this sterilization space chamber is within 30 mm, forming a linear grating GUV LED flowing air and fluid sterilization space device 20.

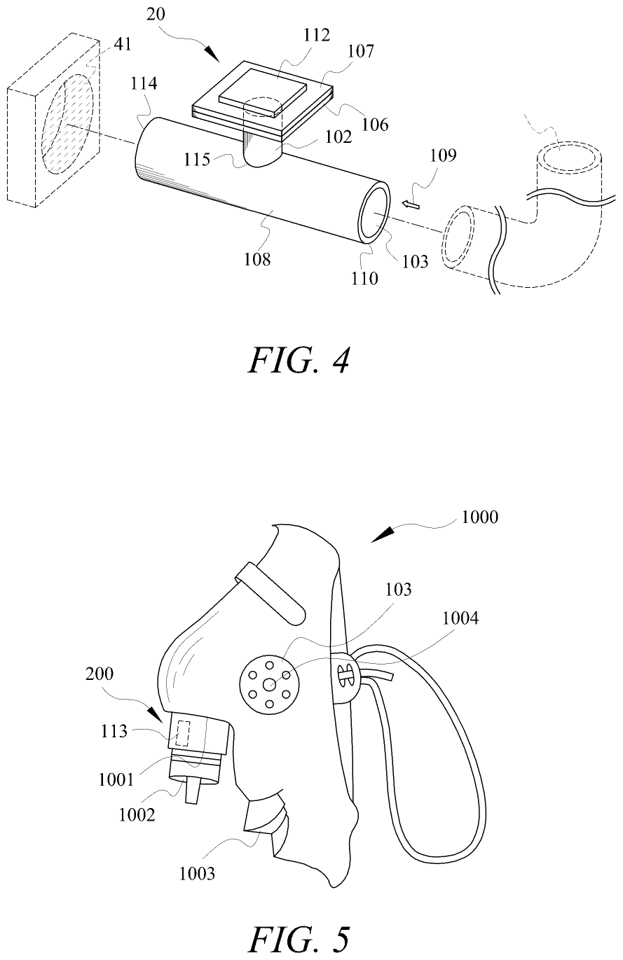

Referring also to FIG. 4, this embodiment is a schematic diagram of the application of the linear grating GUV LED flowing air and fluid sterilization space device 20. By installing the linear grating GUV LED flowing air and fluid sterilization space device 20 between the air intake 42 and the air outlet window 41 of a car or aircraft air conditioning system, it can serve as an air sterilization device for cars or aircraft.

Referring also to FIG. 5, this embodiment is generally similar to embodiment 4, but differs in that the passive multi-linear grating GUV LED flowing air and fluid sterilization space device 20 has a battery added within its frame for power supply, forming a passive sterilization space device 200. It is installed in non-breathable protective cover 1000 made of organic materials, such as masks, nose covers, or face shields that already have air supply. It is embedded near the nose position, with its exterior protruding and exposed at the opening 1001 of the protective cover 1000, and an exhaust port 1002 left at the end. The linear grating GUV LED flowing air and fluid sterilization space device 20 has a battery 113 power device added inside its outer frame 111, illuminating the linear grating GUV LED package 10. The package irradiates the air entering the sterilization space chamber 103 from the side, and the waste gas is discharged through the gas exhaust port 1002. The sterilization space chamber 103 has an air inlet 1004 to introduce air and create positive pressure inside the protective cover 1000. Near the mouth area, there is a crumpled air expansion tank 1003, providing a buffer function for rapid gas expansion during coughing. This serves to process exhaled air containing pathogens for sterilization, preventing infection of others in the vicinity. It is suitable for use by patients with infectious diseases, or for workers who need to talk while preventing infection.

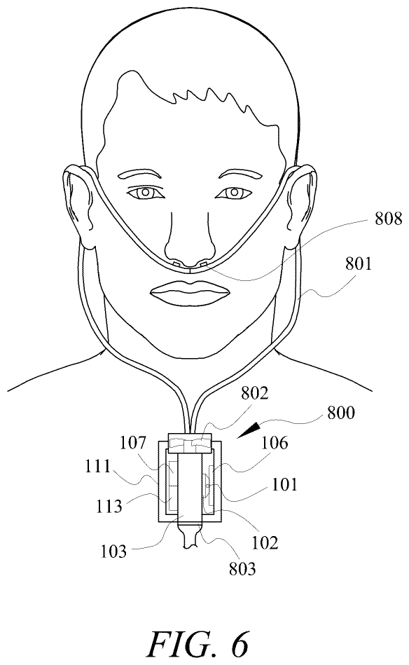

Referring to the embodiment in FIG. 6, which also includes an active sterilization space device 800. This active sterilization space device 800 includes an active fan 802 and an air filter 803, powered by a battery 113 installed inside the outer frame 111, placed on the same side or the other side. It also consists of a GUV LED fixed circuit board 106, a power control PCB board 107, and air/fluid sterilization space chamber 103. One end of the sterilization space chamber 103 is connected to the distal end of a nasal cannula 801, while the fan 802 is positioned between the sterilization space chamber 103 and the nasal cannula 801. At the other end of the sterilization space chamber 103, an air filter 803 is installed to filter out microparticles in the air. The fan 802 actively draws in air, which flows through and is exposed to the linear grating GUV LED package 10. The airflow then passes through the sterilization space chamber 103 for flowing air sterilization and disinfection. The sterilized clean air is then delivered through the nasal cannula 801 to near the wearer's nostrils, and is guided out through the air vent 808 to provide the wearer with clean air to breathe, forming an active flowing air sterilization space device.

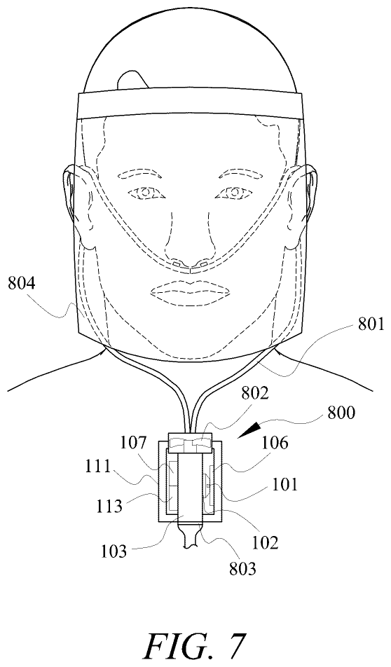

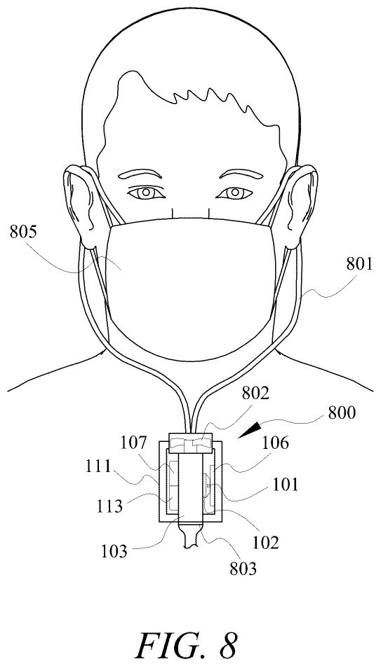

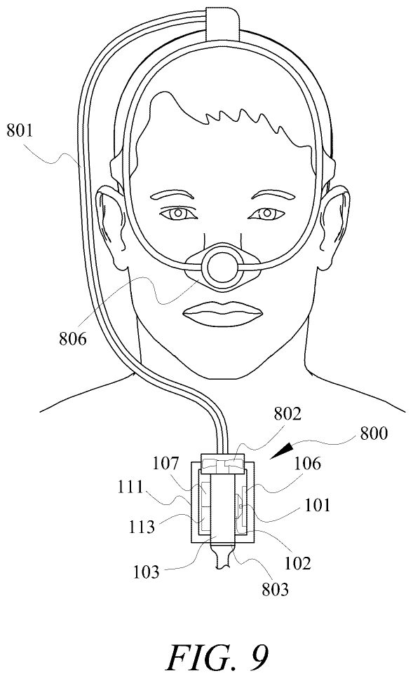

Referring also to FIGS. 7 to 9, these embodiments are generally similar to the embodiment of FIG. 6. As shown in FIG. 7, the clean air after sterilization is fed into the vicinity of the nostrils inside a face mask 804 through the nasal cannula 801, creating a positive pressure inside the face mask 804; as shown in FIG. 7, the clean air after sterilization is fed into the vicinity of the nostrils inside an oral mask 805 through the nasal cannula 801, creating a positive pressure inside the oral mask 805; and as shown in FIG. 9, the clean air after sterilization is fed into the nostrils inside a nasal mask 806 through the nasal cannula 801, creating a positive pressure inside the nasal mask 806.

In these embodiments as shown in FIGS. 7 to 9, by forming a positive pressure inside the face mask 804, the oral mask 805, the nose mask 806, etc., the wearer can breathe more easily, and the air is sterilized by the linear grating GUV LED package 10 before the wearer of the face mask 804, the oral mask 805, the nasal mask 806 inhales the air. The treated clean air is fed into the face mask 804, the oral mask 805, the nasal mask 806 to form a positive pressure space, assisting the person wearing this face mask 804, the oral mask 805, or the nasal mask 806 in inhaling and exhaling. During inhalation, positive pressure oxygen can easily enter the lungs and the person will not feel dizzy due to the lack of oxygen in blood; and during exhalation, exhausted gas can be easily diffused out because of the positive pressure, carbon dioxide, together with steam and hot gas, are also quickly diffused out of the oral mask. When the person inhales again, there is more oxygen and less carbon dioxide, and there is no warm and humid air. As a result, the blood oxygen content is high. Because of the positive pressure, dirty air from outside is not easy to enter the face mask 804, the oral mask 805, or the nasal mask 806, and the wearer is protected from viral and bacterial infections. The above is the use of the positive-pressure safety face mask 804, the oral mask 805, or the nasal mask 806 of the present invention. The fluid sterilization space device further includes other head masks or respirators, etc.

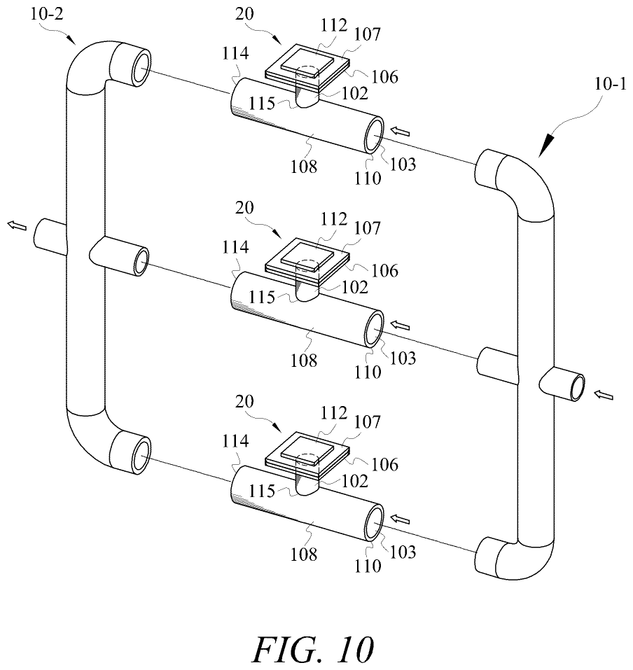

Referring to FIG. 10, it shows a schematic diagram of an embodiment of the linear grating GUV LED flowing air and fluid sterilization space device 20 with parallel high-capacity outputs of the present invention, in which inlets and outlets of the multi-linear grating GUV LED flowing air and fluid sterilization space device 20 described above are connected with multi-way tube joint modules 10-1 and 10-2, thereby increasing the flowing air and fluid processing capacity.

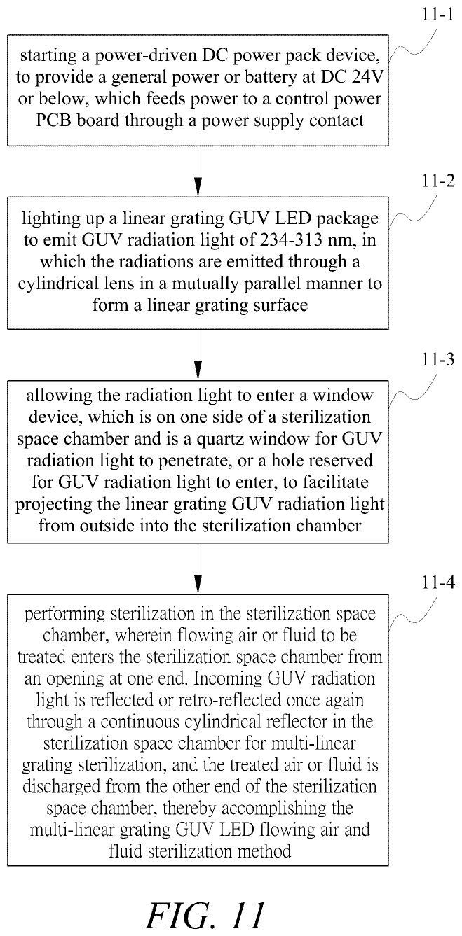

Referring to FIG. 11, it shows an embodiment of a linear grating GUV LED flowing air and fluid sterilization method of the present invention, including the steps of:
11-1, starting a direct current (DC) power pack device, where the power pack device may be a general power supply or a battery 113 under 24 V, that feeds power to a control power PCB board 107 through a power supply contact 112;
11-2, linear grating GUV LED package: delivering power by the control power PCB 107 to start and light up a linear grating GUV LED package 10, where the linear grating GUV LED package 10 emits linear grating GUV radiations of 234-313 nm through a cylindrical lens, in which radiations are emitted in a mutually parallel manner to form a linear grating surface;
11-3, radiation entry window device: the window device is on the side of the sterilization space chamber 103. It can be a quartz window 115 that allows GUV LED to penetrate through, or a pre-reserved hole 116 for GUV LED radiation to enter. The radiation is irradiated from the outside towards the inside;
11-4, sterilization space chamber: the radiation enters the sterilization space chamber 103, and is reflected again by cylindrical reflector on the inner surface, creating multiple linear grating reflections and counter-reflections. The flowing air and fluid to be treated enters the sterilization space chamber 103 from one end in the flow direction 109, undergoes sterilization exposure treatment, and the sterilized air and fluid is discharged from the outlet end 114 of the sterilization space chamber 103.

The multi-linear grating GUV LED flowing air and fluid sterilization method of the present invention is accomplished by utilizing a, the DC power pack device, b, the linear grating GUV LED package, c, the window device for radiations to enter, and d, the sterilization space chamber device.

While the preferred embodiments of the present invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A multi-linear grating GUV LED flowing air and fluid sterilization method, comprising the steps of:
(1) starting a power-driven DC power pack device, to provide a general power or battery at DC 24V or below, which feeds power to a control power PCB board through a power supply contact;
(2) lighting up a linear grating GUV LED package to emit GUV radiation light of 234-313 nm, in which the radiations are emitted through a cylindrical lens in a mutually parallel manner to form a linear grating surface;
(3) allowing the radiation light to enter a window device, which is on one side of a sterilization space chamber and is a quartz window for GUV radiation light to penetrate, or a hole reserved for GUV radiation light to enter, to facilitate projecting the linear grating GUV radiation light from outside into the sterilization chamber; and
(4) performing sterilization in the sterilization space chamber, wherein flowing air or fluid to be treated enters the sterilization space chamber from an opening at one end. Incoming GUV radiation light is reflected or retro-reflected once again through a continuous cylindrical reflector in the sterilization space chamber for multi-linear grating sterilization, and the treated air or fluid is discharged from the other end of the sterilization space chamber, thereby accomplishing the multi-linear grating GUV LED flowing air and fluid sterilization method.

2. A multi-linear grating GUV LED flowing air and fluid sterilization space device, at least comprising:
a. a DC power pack device, comprising a general power supply or a battery power supply under DC 24V, and being used as a power pack of a PCB board for power control;
b. a linear grating GUV LED package which emits ultraviolet rays of 234-313 nm, with the LED packages encapsulated with end-face arc-shaped cylindrical lenses, which can project linear grating surface radiation;
c. a window for radiation light to enter, which is on one side or the other side of the sterilization space chamber, to project the radiation light from outside to inside into a quartz window or a hole of the sterilization chamber; and
d. a sterilization space chamber, which has openings at both ends, with one of concave cylindrical reflector or continuous arc-shaped convex reflector, or continuous arc-shaped concave reflector forming the inner surface of the sterilization space chamber. It has a window reserved on the side for the radiations to enter, in which the hole has an inner diameter of less than 30 mm.

3. The multi-linear grating GUV LED flowing air and fluid sterilization space device of claim 2, wherein the linear grating GUV LED package is an LED including ultraviolet rays of 234-313 nm, and is encapsulated with one of cylindrical lenses having a concave arc-shaped cylindrical shape, a convex arc-shaped cylindrical shape, a continuous concave arc-shaped shape, or a continuous convex arc-shaped cylindrical shape at an end face; and the linear grating GUV LED package is composed of an arc-shaped cylindrical lens by using one of ultraviolet transmitting materials, such as quartz, a fluorinated polymer, polydimethylsiloxane, and polyimide.

4. The multi-linear grating GUV LED flowing air and fluid sterilization space device of claim 3, wherein the sterilization space device comprises at least one multi-linear grating GUV LED package; and the multi-linear grating GUV LED packages are mounted in a window on one side or a different side of the sterilization space chamber, serving as a GUV radiation source of the flowing air and fluid sterilization space device.

5. The multi-linear grating GUV LED flowing air and fluid sterilization space device of claim 2, wherein the sterilization space chamber is made of a hollow quartz tube with openings at both ends. The outer surface of the quartz tube is coated with a highly reflective aluminum layer, or is vacuum-plated with an aluminum reflective layer A channel of transparent window is reserved for the linear grating GUV LED sterilization package. There is one or more windows or hole channels, allowing radiation to enter the flowing air and fluid sterilization space chamber with an inner diameter of less than 30 mm.

6. The multi-linear grating GUV LED fluid sterilization space device of claim 2, wherein the sterilization space chamber is made from metal aluminum or tetrafluoroethylene with a square or circular inner surface, and is formed with continuous convex arc-shaped cylindrical reflector or continuous concave arc-shaped cylindrical reflector on the inner surface of the sterilization space chamber; the sterilization space chamber has a hole channel reserved as an irradiation window of the linear grating GUV LED package; and the sterilization space chamber has openings at both ends with an inner diameter of less than 30 mm, so that flowing air or fluid flows through the sterilization space chamber for radiation sterilization.

7. The multi-linear grating GUV LED flowing air and fluid sterilization space device of claim 6, wherein the sterilization space chamber is made of metal aluminum or tetrafluoroethylene material as a hollow chamber with openings at both ends. A channel hole is reserved on the side for the linear grating GUV LED sterilization package; a transparent hollow quartz tube or hollow fluoropolymer tube with an inner diameter of less than 30 mm is inserted in the middle, serving as a flowing air and fluid sterilization space chamber.

8. The multi-linear grating GUV LED flowing air and fluid sterilization space device of claim 5, wherein the sterilization space chamber is made of a hollow quartz tube or hollow fluoropolymer tube coated with high-reflective aliuminium, and is thus used as a sterilization device for water, sewage, wastewater, and so on.

9. The multi-linear grating GUV LED flowing air and fluid sterilization space device of claim 7, wherein the sterilization space chamber is made of a hollow quartz tube or hollow fluoropolymer tube coated with high-reflective aliuminium, and is thus used as a sterilization device for water, sewage, wastewater, and so on.

10. The multi-linear grating GUV LED flowing air and fluid sterilization space device of claim 6, wherein it comprises a battery device in the outer frame and is powered by the battery device, serving as a passive multi-linear grating GUV LED flowing and air fluid sterilization space device.

11. The multi-linear grating GUV LED flowing air and fluid sterilization space device of claim 10, wherein it further includes an air feeding device provided on one end face and a filter mesh on the other end face; a battery is reserved in the outer frame as a power supply device, which forms an active multi-linear grating GUV LED flowing and air fluid sterilization space device.

12. The active multi-linear grating GUV LED flowing air and fluid sterilization space device of claim 11, wherein a nasal cannula is further provided to connect and deliver the sterilized air through the nasal cannula to the nasal breathing opening of a face mask, mouth mask, nose mask, head cover, or respirator, forming a positive pressure protective mask device.

13. The passive multi-linear grating GUV LED flowing air and fluid sterilization space device of claim 10, wherein the fluid sterilization space device is set at the air outlet end of a non-breathable protective cover. The protective cover has expandable pleats near the mouth area to absorb the sudden increase of gas due to coughing. The used air inside the protective cover is first processed through the passive GUV LED flowing air and fluid sterilization space chamber, and after sterilization, the gas leaves the protective cover device through the air outlet.

14. The multi-linear grating GUV LED flowing air and fluid sterilization space device of claim 2, wherein it further connects multiple linear grating GUV LED fluid sterilization space devices in parallel at their inlets and outlets using an externally connected multi-port connector module, forming a high-capacity output device.
